(19) United States
(12) Patent Application Publication
(10) Pub. No.: US 2011/0137256 A1
(43) Pub. Date: Jun. 9, 2011

(54) FIXATION CATHETER

(75) Inventors: LeVoy Haight, West Jordan, UT (US); Kevin Gordon, American Fork, UT (US); Warren Harris, Lehi, UT (US); Corey Collins, Riverton, UT (US)

(73) Assignee: Upside Services, LLC, Salt Lake City, UT (US)

(21) Appl. No.: 12/632,605

(22) Filed: Dec. 7, 2009

(51) Int. Cl. A61M 25/04

(52) U.S. Cl. 604/175

(57) ABSTRACT

A new and improved catheter for administrating or withdrawing a substance from the human body which can temporarily attached to the tissue site. In one embodiment a filament is attached to the outside of the catheter allowing for ease of attachment and removal. In one embodiment the catheter has multiple holes to allow uniform bathing of the tissue site.

### BACKGROUND OF THE INVENTION

The present invention relates to a tube or catheter, and more particularly to a tube or catheter, which can be temporarily attached to a tissue site within the human body for infusion or withdrawal of a substance from the body.

Conventional technology requires that the suture used to attach the catheter to the tissue run through the catheter tube or be placed in a separate tube attached to the catheter. In both cases, the attachment process is limited because the knot used to create the suture loop cannot pass through the inside of the catheter. This limits the ability to position the catheter once a knot has been tied. Running the suture through the inside of the catheter makes removal of the catheter more difficult because both sides of the knot must be cut since the knot will not pass though the inside of the catheter.

Further, conventional catheters provide a single opening to bath tissue with fluid or extract fluid. This reduces the uniformity of the bathing process because more liquid will be concentrated at the tip of the catheter. When an area requires a fluid bath, fluid is wasted because more fluid will concentrate at the tip of the catheter instead of evenly flowing across the entire area requiring a bath.

A catheter that is simple to attach and remove is needed. Further a catheter that can uniformly bath an area is needed.

### SUMMARY OF THE INVENTION

Embodiments of a system are described. In one embodiment, the apparatus is a catheter comprising a hollow tube with a proximal end and a distal end and a filament with a first end and a second end. The filament first end is coupled to the outside surface of the proximate end of the hollow tube and the filament second end is coupled to the outside surface of the distal end of the hollow tube. Other embodiments of the apparatus are also described.

Embodiments of an apparatus are also described. In one embodiment, the apparatus is a catheter comprising a hollow tube with a proximal end and a distal end with a hole through the side of the hollow tube. The catheter includes a filament with a first end and a second end wherein the filament first end is coupled to the outside surface of the proximate end of the tube and the filament threaded through the hole to an inside of the catheter and threaded through the distal end of the tube to an outside of the catheter. The filament second end is coupled to the outside surface of the distal end of the tube. Other embodiments of the apparatus are also described.

Embodiments of a method are also described. In one embodiment, the method is a method for using a catheter comprising attaching a filament to an outside surface of a proximate end of the catheter and attaching the filament to body tissue. Attaching the filament to the outside surface of a distal end of the catheter and administering fluids through the catheter. Other embodiments of the method are also described.

Embodiments of a method are also described. In one embodiment the method is a method for bathing biological tissue and attaching a catheter to biological tissue. Introducing a fluid into the catheter and the fluid exiting more than one opening located near the biological tissue.

Other aspects and advantages of embodiments of the present invention will become apparent from the following detailed description, taken in conjunction with the accompanying drawings, illustrated by way of example of the principles of the invention.

### BRIEF DESCRIPTION OF THE DRAWINGS

FIG. 1 depicts a schematic diagram of one embodiment of a catheter.

FIG. 2 depicts a schematic diagram of one embodiment of a catheter.

FIG. 3 depicts a schematic diagram of one embodiment of a catheter.

FIG. 4 depicts a schematic diagram of one embodiment of a catheter.

FIG. 5 depicts a schematic diagram of a method for using a catheter.

FIG. 6 depicts a schematic diagram of a method for bathing biological tissue.

FIG. 7 depicts a schematic diagram of one embodiment of a catheter.

FIG. 8 depicts a schematic diagram of one embodiment of a catheter.

Throughout the description, similar reference numbers may be used to identify similar elements.

### DETAILED DESCRIPTION

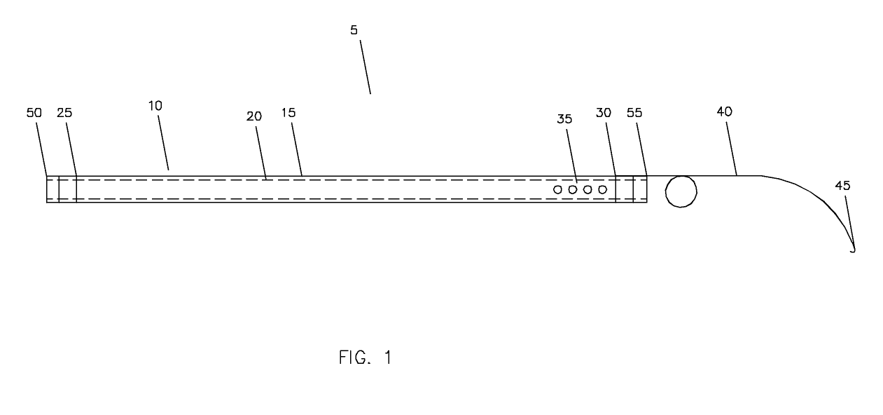

FIG. 1 depicts a schematic diagram of one embodiment of a catheter 5 having a hollow tube 10 with an outside surface 15 and an inside surface 20. A first band 25 is attached to the outside surface 15 of the hollow tube 10 and a second band 30 is attached to the outside surface 15 of the hollow tube 10. Holes 35 are located near second band 30. Holes 35 may extend through hollow tube 10. In one embodiment holes 35 are not used. Hollow tube 10 has proximal end 55 and distal end 50. Filament 40 is attached to second band 30 on one end and attached to needle 45 on the other end.

Hollow tube 10 can be made out of any suitable bio compatible material such as polyurethane, silicone, elastomer, Teflon, Nylon, Pebax, etc. In one embodiment hollow tube 10 is 36 inches long and is 19 gauge catheter with 0.042 inch outside diameter and 0.035 inches inside diameter. First band 25 and second band 30 can be made of any suitable bio compatible material that is rigid enough to prevent hollow tube 10 from collapsing. In one embodiment first band 25 and second band 30 has an outside diameter of 0.052 inches and an inside diameter of 0.042 inches. In one embodiment second band 30 is located 0.039 inches from the proximate end 55 of hollow tube 10 and first band 25 is located 7 or 8 inches from the proximate end 55 of hollow tube 10.

Filament 40 may be made of any suitable bio compatible material and in one embodiment Filament is made of polyester monofilament. In one embodiment filament 40 is 0.005 inch medical grade filament that is 10 inches long. In one embodiment filament 40 is made from 3.0 or 5.0 silk about 30 inches long. Filament 40 may be attached to second band 30 by bio compatible tape, glue or by tying filament 40 to first band 30. Needle 45 may be made of any suitable bio compatible material that is rigid enough to pierce body tissue. In one embodiment needle 45 is not used.

Catheter 5 is used by attaching filament 40 through biological material such as body tissue, and placing hollow tube 10 near the attachment site. Filament 40 is then secured by tying filament 40 around hollow tube 10 and bellow first band 25 preventing filament 40 from moving toward proximate end 55. In one embodiment filament 40 may be taped to first band 25. The body tissue may then be bathed by fluid passing through hollow tube 10 and exit out holes 35 or a hole located in the proximate end 55 of hollow tube 10. Additionally, the body tissue may be drained by fluid entering holes 35 or a hole located in the proximate end 55. In one embodiment holes 35 are 1/64 inches in diameter.

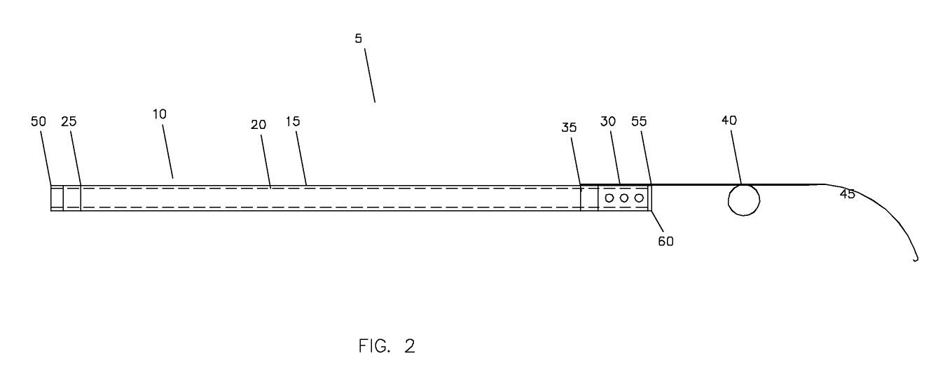

FIG. 2 depicts a schematic diagram of one embodiment of a catheter 5 having cap 60 placed over proximal end 55 to prevent fluid from entering or exiting the end of the proximal end 55 of the hollow tube 10.

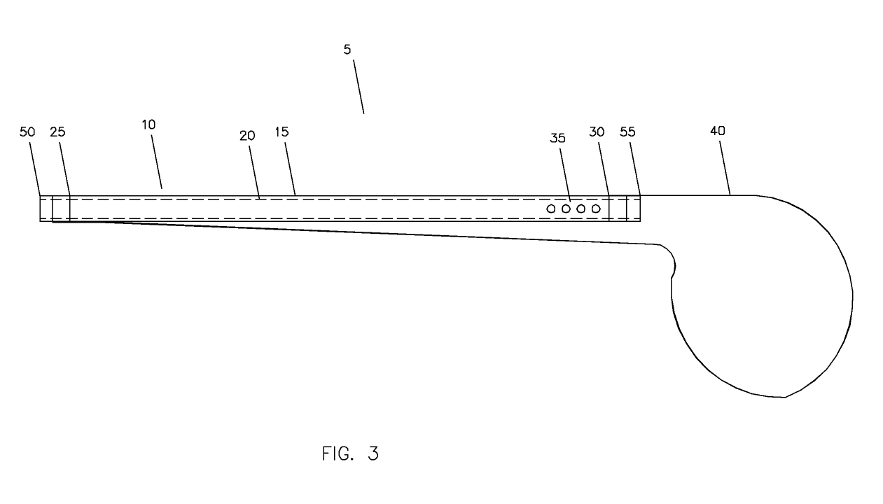

FIG. 3 depicts a schematic diagram of one embodiment of a catheter 5 showing how filament 40 is tied below first band 25 or taped to first band 25 to secure hollow tube 10 in place.

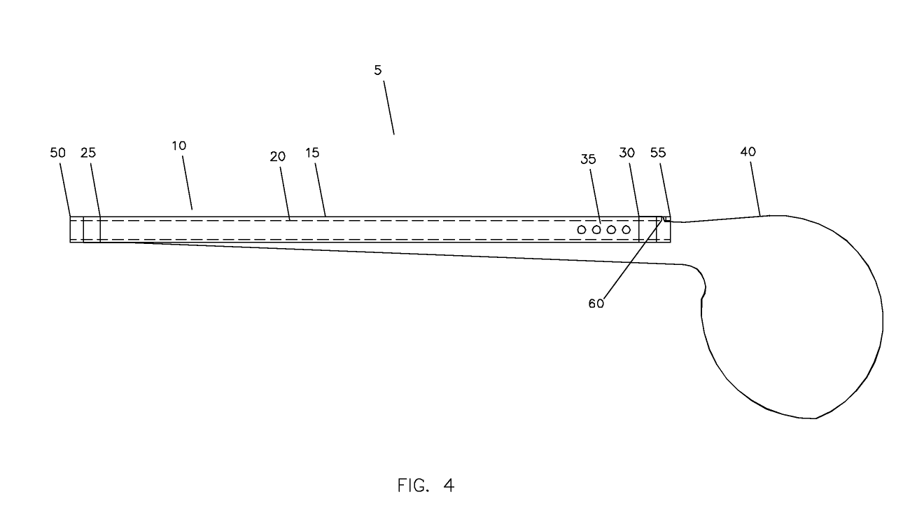

FIG. 4 depicts a schematic diagram of one embodiment of a catheter 5 having hole 60 located above second band 30 and filament 40 is attached to second band 30 and passes through hole 60 to the inside of hollow tube 10. Filament 40 then passes through proximate end 60 of hollow tube 40 and is tied below first band 25 or taped to first band 25.

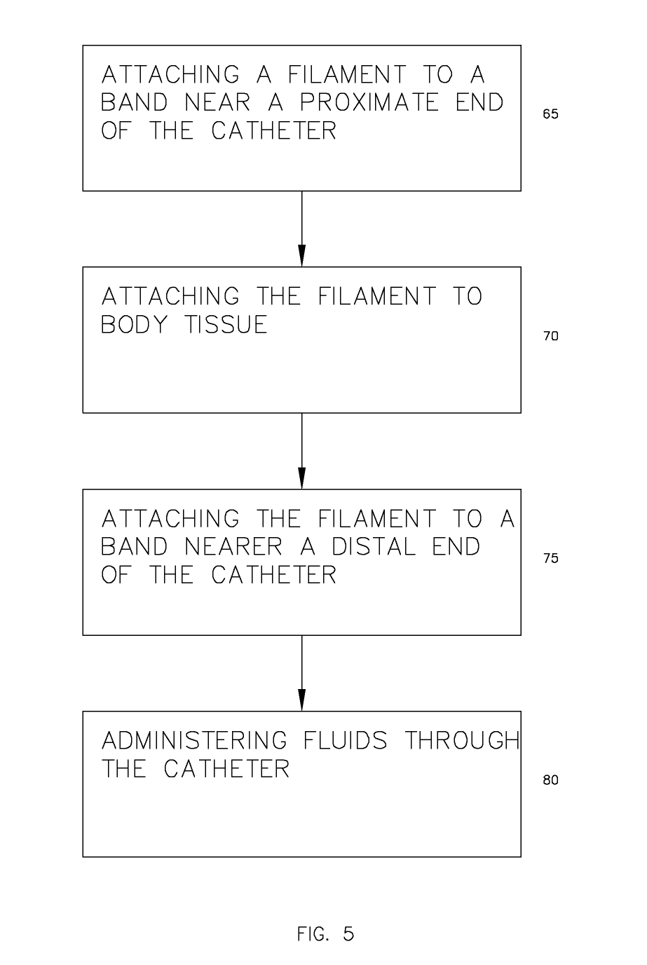

FIG. 5 depicts a schematic diagram of a method for using a catheter including steps 65 through 80. The steps comprising attaching the filament 40 to the outside surface 15 of a proximate end 55 of the catheter. Then attaching the filament 40 to body tissue and attaching the filament 40 to the outside surface 15 of a distal end 50 of the catheter 5 and administering fluids through the catheter 5.

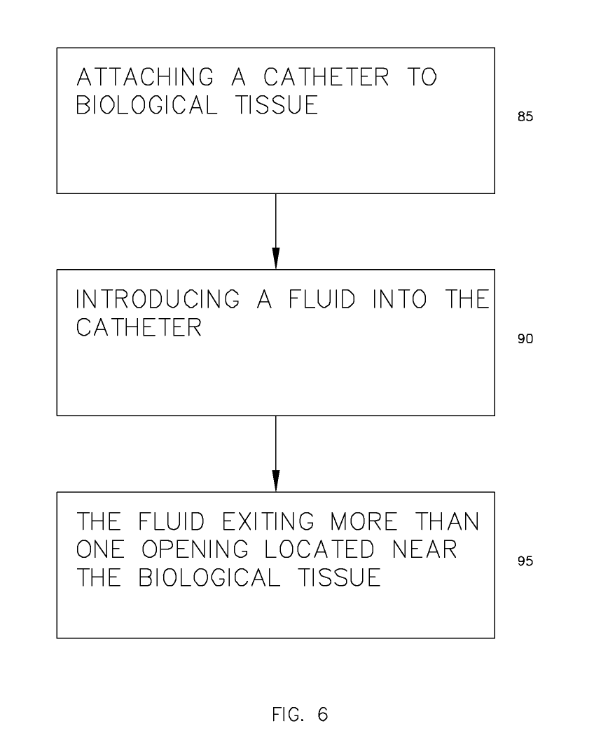

FIG. 6 depicts a schematic diagram of a method for bathing biological tissue including steps 85 through 95. The steps comprising attaching the catheter 5 to biological tissue and introducing a fluid into the catheter 5. Further, the fluid exiting more than one opening 35 located near proximal end 55 of the catheter 5.

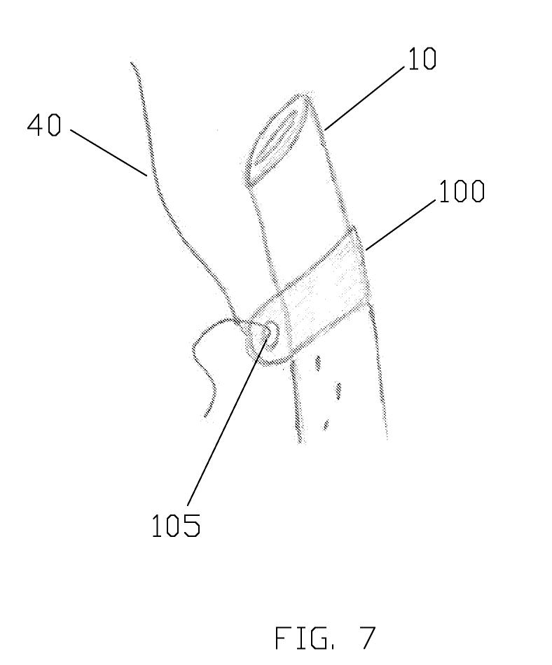

FIG. 7 depicts a schematic diagram of one embodiment of a catheter. Tab 100 is coupled to hollow tube 10 and filament 40. Filament 40 attached to tab 100 by tying a knot through tab hole 105. This embodiment allows the person attaching the hollow tub to attach filament 40 to hollow tube 10. This allows a person to select the type filament they would like to use instead of having the filament selected by the manufacture. Additionally, this can reduce production costs by eliminating the need to attach filament 40 during the manufacturing of the catheter.

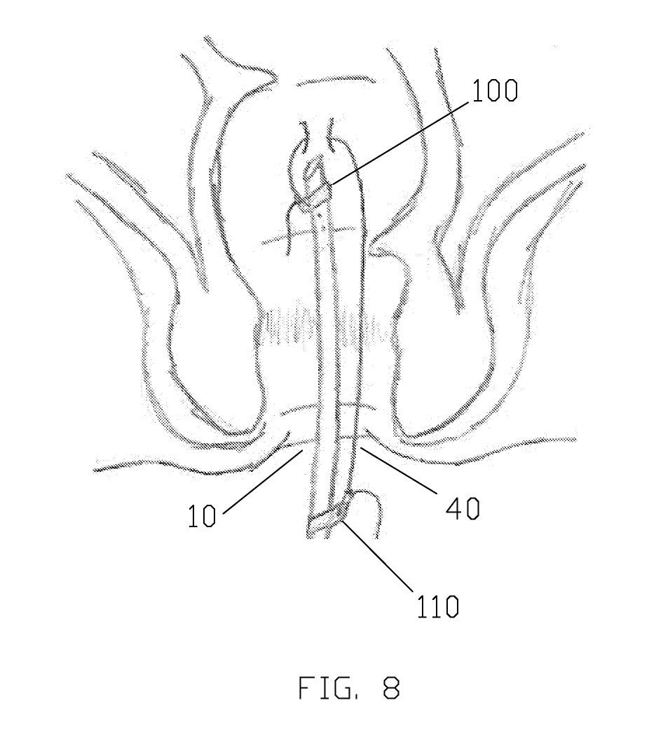

FIG. 8 depicts a schematic diagram of one embodiment of a catheter. Filament 40 is tied to tab 100 and to tie-off tab 110. This prevents hollow tube 10 from moving from the attachment site. Hollow tube 10 can be removed by cutting filament 40 between tab 100 and tie-off tab 110 and pulling on hollow tube 10.

Several advantages are realized with the above described embodiments. The multiple holes allow for uniform bathing of body tissue by more evenly distributing a fluid. Attaching a filament to the outside of the hollow tube eliminates knots in the filament that can interfere with the operation of the catheter by blocking fluid flow though the tube. Additionally, the catheter can be easily removed without the possibility of damaging body tissue by passing a knot through the body tissue attachment location. Cutting the filament at the base of the catheter assures no knots are in the filament as the filament is removed. Further, keeping the filament on the outside of the catheter eliminates the need to thread the filament through the hollow tube.

Although the operations of the method(s) herein are shown and described in a particular order, the order of the operations of each method may be altered so that certain operations may be performed in an inverse order or so that certain operations may be performed, at least in part, concurrently with other operations. In another embodiment, instructions or sub-operations of distinct operations may be implemented in an intermittent and/or alternating manner.

Although specific embodiments of the invention have been described and illustrated, the invention is not to be limited to the specific forms or arrangements of parts so described and illustrated. The scope of the invention is to be defined by the claims appended hereto and their equivalents.

What is claimed is:

1. A catheter comprising:
a hollow tube with a proximal end and a distal end; and
a filament with a first end and a second end wherein the filament first end is coupled to the outside surface of the proximate end of the hollow tube and the filament second end is coupled to the outside surface of the distal end of the hollow tube.

2. The catheter of claim 1 further comprising one or more side holes in the hollow tube.

3. The catheter of claim 2 wherein the proximal end of the hollow tube is closed.

4. The catheter of claim 1 further comprising a first band coupled to the proximate end of the hollow tube.

5. The catheter of claim 4 wherein the filament first end is coupled to the first band

6. The catheter of claim 4 wherein the filament first end is glued to the first band

7. The catheter of claim 4 further comprising a second band coupled to the distal end of the hollow tube.

8. The catheter of claim 7 wherein the filament second end is coupled to the second band.

9. The catheter of claim 7 wherein the filament second end is taped to the second band.

10. The catheter of claim 7 wherein the filament second end is tide around the hollow tube below the second band.

11. A method for using a catheter comprising:
coupling a filament to an outside surface of a proximate end of the catheter;
attaching the filament to body tissue;
coupling the filament to the outside surface of a distal end of the catheter; and
administering fluids through the catheter.

12. The method of claim 11 wherein the filament is tied around the catheter.

13. The method of claim 12 wherein the filament is taped to the catheter

14. A catheter comprising:
a hollow tube with a proximal end and a distal end;
a hole through the side of the hollow tube;
a filament with a first end and a second end wherein the filament first end is coupled to the outside surface of the proximate end of the tube, the filament threaded through the hole to an inside of the catheter and threaded through the proximal end of the tube to an outside of the catheter; and
the filament second end coupled to the outside surface of the distal end of the tube.

15. The catheter of claim 14 further comprising more than one hole in the catheter located near the distal end of the catheter.

16. The catheter of claim 14 further comprising a first band coupled to the proximate end of the hollow tube.

17. The catheter of claim 16 wherein the filament first end is coupled to the first band.

18. The catheter of claim 17 further comprising a second band coupled to the distal end of the hollow tube and filament second end tied around the hollow tube.

19. A method for bathing biological tissue:
attaching a catheter to biological tissue;
introducing a fluid into the catheter; and
the fluid exiting more than one openings in the catheter located near a proximal end of the catheter

20. The method of claim 19 wherein the number of openings is more than 2.
